(19) United States
(12) Patent Application Publication
(10) Pub. No.: US 2010/0234744 A1
(43) Pub. Date: Sep. 16, 2010

(54) BLOOD VESSEL SENESCENCE DETECTION SYSTEM

(75) Inventor: Reiji Kawada, Iwakuni-shi (JP)

Correspondence Address: SUGHRUE MION, PLLC, 2100 PENNSYLVANIA AVENUE, N.W., SUITE 800, WASHINGTON, DC, 20037, US

(73) Assignee: Retinal Information Diagnosis Research Institute Inc., Iwakuni-shi Yamaguchi (JP)

(21) Appl. No.: 12/301,137

(22) PCT Filed: May 15, 2007
(86) PCT No.: PCT/JP2007/059987

§ 371 Date: Feb. 18, 2009

(30) Foreign Application Priority Data
May 16, 2006 (JP) 2006 136374

(51) Int. Cl. A61B 5/02

(52) U.S. Cl. 600/500

(57) ABSTRACT

A blood vessel senescence detection system characterized in that a means for detecting volume pulse waves of paired eyeground artery and vein traveling in the same direction, and a means for detecting peripheral vascular resistance by using waveform differences between the both pulse waves is provided. According to the detection system, the degree of senescence of the capillary of the body, and furthermore the degree of the whole cardiovascular system of the body, can be detected, for example, as a rate of change with time or a vascular age, by using the waveform difference between the volume pulse wave of the eyeground artery and the volume pulse wave of the eyeground vein as a parameter.

### TECHNICAL FIELD

The present invention relates to a blood vessel senescence detection system. Meanwhile, the progression of the blood vessel senescence is typically expressed as increase of a rate of change of arteriosclerosis with time.

### BACKGROUND ART

The circulation of the blood in the human body is the closed circuit course that assumes a heart the main pump. However, in the closed circuit, pump action by the blood vessel pulsating with the great and medium blood vessel, which means the Windkessel phenomenon, contributes greatly than it is imagined so far.

There are two constitutions of the pulse pressure waveform in the aortic valve opening which valve has the greatest blood vessel diameter. One is a drive pressure wave (a front ingredient in the presystolic period) sent off directly from the heart. The other is a reflection pressure wave (namely a backward ingredient in the presystolic period) caused by peripheral vessel resistances.

It is supposed that the reflection pressure wave of the latter is very influential in the starting of the mechanism of the blood pressure rise in the general blood pressure measurement.

In fact, “mean blood pressure” is a pressure generated by steady streaming without the pulsating and increased by sclerosing of the peripheral vessel or by rising of the peripheral vessel resistance from its vasoconstriction. “The pulse pressure” is increased by the sclerosing, namely the falling of the extensibility, of the great blood vessel, which pressure is derived from the blood pressure at a presystolic phase by loss of the one at a diastole phase. Therefore, in a comparatively young time, “mean blood pressure” is increased as the peripheral vessel resistance rises. And, with senescence, “pulse pressure” tends to be increased as the great blood vessel sclerose.

Now β value is used as an index of arteriosclerosis of the great and medium artery. However, an index has not been established yet that indicates the senescence of the peripheral vessel.

Patent document No.1: JP 2003-299621 A
Patent document No.2: WO2004/004556

### DISCLOSURE OF THE INVENTION

### Subject to be Solved by the Invention

However, it is insufficient to grasp only the sclerosing of the great and medium arteries in order to grasp the senescence of the blood vessel truly. In other words the senescence of the peripheral vessel is directly linked to the senescence of the microvessel (the tissular blood capillary) of the internal organs. And it can become a convincing index of the arteriosclerosis of the whole body in its turn. Therefore, it is thought to be essential to detect the senescence of the peripheral vessel in order to grasp the senescence of the blood vessel. When an arterial flow shifts to a peripheral vessel, the reflection pressure wave mentioned above occurs. An increased amplitude of the reflection pressure wave suggests an increased peripheral vessel resistance to the forward bloodstream, namely, a possibility of occlusions or sclerosis of the peripheral vessel. However, the increased amplitude of the reflection pressure wave is only an indirect index, and is easy to change depending on measurement conditions (measurement sites, measurement method). In addition, the reflection pressure wave includes contamination waves in high frequency. Therefore, this is still insufficient for an index to grasp the state of the microvessel (the capillary in tissues) of internal organs.

### Means for Solving the Subject

The present inventor found that, as a result of having examined for solving the problem mentioned above zealously, it could be solved by paying attentions to the eyeground where the system comprising an artery, a capillary (a microvessel (a capillary in tissue)), and a vein can be observed directly with eyes, and to the difference in the waveforms of the eyeground vein and artery, and this difference is used as a direct index of the peripheral vessel resistance.

According to one aspect of the present invention, there is provided a blood vessel senescence detection system comprising: a volume pulse waves detecting means for detecting volume pulse waves of paired eyeground artery and vein traveling in the same direction; and a peripheral vascular resistance detecting means for detecting the peripheral vascular resistance by using waveform difference between the volume pulse wave of the eyeground artery and the volume pulse wave of the eyeground vein as a parameter (hereinafter, the blood vessel senescence detection system may be referred to as “the present detection system”).

In the present invention, an “eyeground artery” means a “retina artery” and an “eyeground vein” means a “retina vein” saying to a medical term. In addition, the “paired eyeground artery and vein traveling in the same direction” means, when an eyeground artery leads to an eyeground vein through a microvessel in an eyeground, the combination of the eyeground artery and the eyeground vein.

When the volume pulse waves of the paired eyeground artery and vein are detected from the outside, the waveform difference between the volume pulse wave of the eyeground artery and the volume pulse wave of the eyeground vein can be grasped by making a so-called blood vessel volume pulse wave figure, which is made by plotting the change in blood vessel width (expansion and reduction) consecutively per tiny unit time (e.g. 0.1 sec to 0.01 sec: the shorter, the better) and grasping the change in the blood vessel width with time. Desired waveform difference can be detected by making this kind of blood vessel volume pulse wave figure for each of the paired eyeground artery and vein traveling in the same direction, comparing these figures and examining the similarity thereof. Generally, the more similar the unit waveforms in the volume pulse wave figures of the paired eyeground artery and vein are, the lower the peripheral vessel resistance is at the microvessel in the eyeground (it means that the blood streams down this peripheral vessel smoothly). On the other hand, the less similar they are, the higher the peripheral vessel resistance is, which means that the blood doesn't stream down the peripheral vessel smoothly.

In one preferable aspect of the present invention, the waveform difference in the volume pulse waves is component difference between an acceleration pulse wave of the eyeground artery and an acceleration pulse wave of the eyeground vein, the acceleration pulse waves being caused by propagation of the volume pulse waves.

The “acceleration pulse wave” is the second temporal differential pulse of the volume pulse wave (The first temporal differential pulse is called “the speed pulse wave”). The speed pulse wave facilitates the recognition of the point of inflection in the volume pulse wave. The acceleration pulse wave expresses a waveform change in front and back of the point of inflection. The acceleration pulse wave is known to include partial waves, e.g., an early presystolic positive wave (a wave), an early presystolic negative wave (b wave), a middle presystolic reascension wave (c wave), a late presystolic re-dropping wave (d wave), and an early diastolic positive wave (e wave). They are measured by measuring the deflection from the baseline to the top of the waveform for each partial wave. The component difference means the difference between the ratio of the sizes on the specific partial waves of the acceleration pulse wave of the eyeground artery and that of the eyeground vein. For example, the component difference between b wave/a wave or d wave/a wave of the acceleration pulse wave obtained from the volume pulse wave of the eyeground artery and b wave/a wave or d wave/a wave of the acceleration pulse wave obtained from the volume pulse wave of the eyeground vein is determined, and, when the difference between them is big as the absolute value, the peripheral vessel resistance of the eyeground is recognized to be high. On the other hand, when the difference is small, the peripheral vessel resistance is recognized to be low.

The waveform difference between the volume pulse wave of the eyeground artery and the volume pulse wave of the eyeground vein can be detected on the basis of the eyeground artery image and the eyeground vein image captured by an eyeground camera. That is, the images of paired eyeground artery and vein traveling in the same direction are obtained by an eyeground camera, and the waveform difference can be detected on the basis of a change with time (in very short or minimal time) in each of the diameters of the eyeground artery and vein in the images. Such images may be consecutive (moving image) or still, but consecutive images are preferable.

The volume pulse waves of the eyeground artery and vein can be detected on the basis of the ultrasound Doppler method (a method by applying ultrasound diagnosis to a blood vessel and determining a component of transmission velocity of the bloodstream on the basis of the Doppler effects observed in the reflection wave: for example, the ultrasound color Doppler method). It is preferable that Doppler in such ultrasound Doppler is used for an eyeground artery. In addition, the laser Doppler method can be used in place of the ultrasound Doppler method. The laser Doppler is a method using a measurement device for exposing a laser beam toward a blood corpuscle cell streaming down a blood vessel, and measuring blood flow rate. In this method, the faster the blood streams, the redder the indication of the device turns, and the volume pulse wave can be detected by detecting expansion and presystolic of the eyeground artery.

The above-mentioned eyeground camera, or ultrasound or laser Doppler method can be used together to detect the volume pulse waves. For example, the volume pulse wave of the eyeground artery is detected by using the ultrasound Doppler method or laser Doppler method, and the volume pulse wave of the eyeground vein is detected by using the eyeground camera.

In another preferable aspect of the present invention, the waveform difference between the volume pulse waves as described above is detected in synchronization with a biosignal selected from the group consisting of an electrocardiographic signal, a fingertip sphygmographic signal, an eyepit sphygmographic signal, a temporal artery sphygmographic signal, a carotid artery sphygmographic signal, and a pulse oximeter signal. The pulse oximeter signal can be detected as pulse waves at earlaps or fingertips, as described below. That is, the fingertip sphygmographic signal is also a biosignal which should be understood as one mode of the pulse oximeter signal.

No particular limitations are imposed on the electrocardiographic signal detection means, so long as the electrocardiographic signal can be precisely detected. The electrocardiographic signal may be selected arbitrarily. The electrocardiographic signal may be any signal selected from established patterns on an electrocardiogram, and is preferably a signal which can be identified as an established waveform pattern on an electrocardiogram. Specifically, the electrocardiographic signal may be any waveform pattern selected from among a P wave, a Q wave, an R wave, an S wave, and a T wave. However, an R wave which is the pattern signal detected when blood is fed from the heart to the entire body, or a T wave which corresponds to recovery of ventricular activation, is preferably and practically employed.

No particular limitations are imposed on the lead for obtaining an electrocardiographic signal from a subject, and the lead may be selected from, for example, “standardized 12-lead ECG.” For example, when an R wave is selected as an electrocardiographic waveform pattern, lead II, lead I, or lead ₐV_{L}, in which the difference in electric potential between the left and right hands of a subject is measured, is preferably employed.

In the present detection system, “an electrocardiogram signal perceiving means” which perceives an electrocardiogram signal detected by the electrocardiogram signal detecting means, takes out a specific pattern signal from the perceived electrocardiogram signal as an electrical signal, and transmits it to the volume pulse waves detecting means, is a matter of choice used as a constituent element in cases of needs for preprocessing the electrocardiogram signal on the assumption that the volume pulse wave is synchronized to the electrocardiogram signal.

As for the fingertip pulse wave, a fingertip is comparatively far from an eyeground, but its measurement is advantageously quite simple and easy. In other words, by applying the principle of the pulse oximeter which is described below, to the bloodstream under the nail of the fingertip, the bloodstream information can be obtained as a two-phase pulse wave signal. The pulse wave signal can also be obtained by the ultrasound or laser Doppler method.

As for the eyepit pulse wave, it can be obtained by measuring the pulsating of the artery at an eyepit and grasping its pulsation wave. As an eyepit is close to eyeground artery and vein, the eyepit pulse wave is extremely advantageous when pursuing the synchronous accuracy. The eyepit pulse wave can be obtained by the intraocular ultrasound or laser Doppler method.

As for the temporal artery pulse wave (temple pulse wave), it can be obtained by measuring the pulsating of the temporal artery and grasping its pulsation wave. As a temporal artery is close to eyeground artery and vein, the temporal artery pulse wave is extremely advantageous when pursuing the synchronous accuracy. The temporal artery pulse wave can be obtained by the ultrasound or laser Doppler method.

As for the carotid artery pulse wave, a carotid artery is close to eyeground artery and vein, and the amplitude of the carotid arterial pulsation is so great that the pulse wave itself is extremely easy to be grasped. The ultrasound or laser Doppler method is preferable for detecting the carotid artery pulse wave, because the amplitude of the carotid arterial pulsation is great.

A pulse oximeter is a measurement machine for simply and easily measuring an arterial oxygen saturation (SpO₂). Exposed by a visible ray (660 nm), the hemoglobins connected with oxygen (oxygenated hemoglobins) appear red, and, the hemoglobins not connected with oxygen (reduced hemoglobins) appear black. Exposed by an infrared ray (940 nm), the oxygenated hemoglobins appear red, and, the reduced hemoglobin appear red. The difference in such color is a phenomenon caused by the fact that absorbance of each hemoglobin is different. When absorption of light (absorbance) is measured through use of such characteristics, the ratio of oxygenated hemoglobins and reduced hemoglobins becomes clear, and the oxygen saturation can be obtained. However, the absorption of the light occurs not only in the artery but also in the vein or other tissues, so it is necessary to distinguish the artery from the vein and other tissues and to measure the arterial oxygen saturation. Because light absorption components in arterial blood changes by pulsating, SpO₂ can be measured by deducting the components without pulsating (the vein and other tissues) from the total light absorption components. SpO₂ can be measured with time by emitting two-wavelength lights (red and infrared lights) in turn at the frequency of several hundred times per second from a light-emitting part of a sensor in a probe of a pulse oximeter, the sensor comprising the light-emitting part and a light-receiving part, and carrying out deducing calculation as described above to obtain signals. SpO₂ actually indicated in a pulse oximeter is a mean value for few seconds, but a “pulse wave signal of a pulse oximeter” used in the present detection system is not such a mean value, but is a pulse wave signal directly provided from the sensor as described above. Since the pulse wave signal is provided as a feeble electrical signal, the purpose of the present detection system can be achieved by synchronizing the electrical signal with the information of the eyeground image.

The sensor for detecting a pulse wave signal of a pulse oximeter includes, as described above, a light-emitting part which emits visible and infrared lights intermittently, and a light-receiving part which has an optical sensing function to receive the transmitted light and its feeble changes, the transmitted light obtained by transmitting the light emitted from the light-emitting part to a detected part such as a fingertip or an earlap, and to convert these changes into feeble changes in an electric current. This feeble change of the electric current is expressed as a “phase changing pulse wave signal.” The pulse wave signal may be subjected to processing as needed, or arithmetic processing of deducting elements of the vein and other tissues as described above or amplifying processing. In addition, an output terminal may be used to perceive and transmit outside only an electrical signal which shows a specific phase.

In the present invention, “in synchronization with” means detecting the volume pulse waves in response to a selected timing on an electrocardiographic signal, a fingertip sphygmographic signal, an eyepit sphygmographic signal, a temporal artery sphygmographic signal, a carotid artery sphygmographic signal, or a pulse oximeter signal. This will enable us to get accurate information on eyeground vessels, more specifically, accurate information on volume pulse waves of paired eyeground artery and vein traveling in the same direction, the information being inevitable to detect the component difference between the volume pulse waves of the eyeground artery and vein.

Detection of an eyeground image which is one of preferable means for detecting the volume pulse waves is typically carried out by a camera having a mechanism capable of taking an analog/digital picture of an eyeground (its specific example is the so called “eyeground camera, which may be an analog camera or an digital camera.). In this case, the shutter is set so as to click in synchronization with a specific pattern of an electrocardiographic signal, a fingertip sphygmographic signal, an eyepit sphygmographic signal, or a temporal artery sphygmographic signal. In view of the fact that the cycle length of the human pulse wave is usually 0.6 to 1.0 second, it is preferable that the shutter clicks once or more per 0.4 second and the difference between the time of crick of the shutter and the time of exposure is as short as possible.

Therefore, it is suitable to use a digital camera with a photographing element such as CCD, which is easy to technically fulfill such conditions. Detection of eyeground images by using a digital video camera, which captures eyeground images continuously as digital image information, is suitable for synchronization with an electrocardiographic signal, a fingertip sphygmographic signal, an eyepit sphygmographic signal, a temporal artery sphygmographic signal, a carotid artery sphygmographic signal, or a pulse oximeter signal, on a computer, as described below.

In the present detection system, the target sites at which the eyeground artery and vein diameters are measured are preferably paired eyeground artery and vein traveling in the same direction in vicinity to the optic papilla, where pulsation of eyeground artery and vein is most remarkably presented.

The eyeground artery and vein diameters may be measured at every target site through direct visual observation of an eyeground image obtained. This measuring process may be automated by providing an eyeground camera, or the eyeground image detection means, with eyeground artery and vein diameter measuring means capable of measuring the eyeground artery and vein diameters at every target site. An example of such eyeground artery and vein diameter measuring means is software in which means for measuring the diameter of an eyeground artery or vein at a target site on the basis of the electronic data of the eyeground image obtained from the video or moving image as described above is programmed. When the aforementioned eyeground image data are processed by use of such software, the eyeground artery and vein diameters can be conveniently and reliably measured with time, and a volume pulse wave figure can be drawn on the basis of the measurement data, to detect the waveform difference between the volume pulse waves of the paired eyeground artery and vein traveling in the same direction. It means that the greater the degree of the waveform difference is, the stronger the resistance to the bloodstream of the forward direction of the capillary in the eyeground is, namely the more stenosed or sclerotized this capillary becomes. A blood vessel system equivalent to a microvasculature system lying between an arteriole and a venula in the human body lies between the paired eyeground artery and vein traveling in the same direction to constitute a closed blood vessel system. The stenosis or sclerotization of this microvasculature system plainly shows a tendency of the stenosis or sclerotization of the microvasculature of the internal organs system level of the human body. Therefore, when the difference between the volume pulse waves of the paired eyeground artery and vain traveling in the same direction is detected, the degree of senescence of the capillary system of the whole human body can be detected by using the difference as an index.

The speed pulse wave and its figure can be obtained by first temporal differentiation of the function constituting the volume pulse wave figure. In case where the speed pulse wave figure is used to detect the waveform difference between paired eyeground artery and vein traveling in the same direction, the degree of senescence of the capillary system can be precisely detected as well as or better than the case where the volume pulse wave figure is used.

The acceleration pulse wave and its figure can be obtained by second temporal differentiation of the function constituting the volume pulse wave figure, as described above. When the acceleration pulse wave figure is used to calculate the component difference between paired eyeground artery and vein traveling in the same direction, the degree of senescence of the capillary system can be detected most precisely by using the component difference as an index.

In the most preferred mode of the present detection system, detection of an eyeground image is performed by use of software which can provide an eyeground image in synchronization with an electrocardiographic signal, a fingertip sphygmographic signal, an eyepit sphygmographic signal, a temporal artery sphygmographic signal, a carotid artery sphygmographic signal, or a pulse oximeter signal, by extracting, on a computer display, a stationary eyeground image in synchronization with an arbitrary electrocardiographic signal, a fingertip sphygmographic signal, an eyepit sphygmographic signal, a temporal artery sphygmographic signal, a carotid artery sphygmographic signal, or a pulse oximeter signal, from a moving eyeground image (hereinafter the software may be referred to as “the present software”).

In this mode, a digital video (DV) camera is used as the eyeground image detection means to capture a motion eyeground image, and the motion eyeground image is input, as digital data, into a computer via, for example, a DV terminal (media converter is also available) and a DV capture card such as IEEE1394 card, EZDV (product of Canopus Co., Ltd.), DVRapter (product of Canopus Co., Ltd.), or DVRex (product of Canopus Co., Ltd.), with an electrocardiographic signal converted into a digital signal by use of, for example, an analog/digital (A/D) converter being input into the computer. Subsequently, the input motion eyeground image data and the input electrocardiographic signal data are combined in parallel so as to synchronize the eyeground image data with the electrocardiographic signal, a fingertip sphygmographic signal, an eyepit sphygmographic signal, a temporal artery sphygmographic signal, a carotid artery sphygmographic signal, or a pulse oximeter signal in the same frame, thereby yielding digital synchronization data of the motion eyeground image data and the electrocardiographic signal, a fingertip sphygmographic signal, an eyepit sphygmographic signal, a temporal artery sphygmographic signal, a carotid arteries sphygmographic signal, or a pulse oximeter signal. The digital synchronization data may be subjected to compression, so long as data required for implementing the present detection system are not lost. Coding such as compression may be performed by means of a coding format such as MPEG or MP3. Compressed or coded data may be used on the present system when partially or completely decompressed by using existing zip-unzip technologies (for example; the zip-unzip technology developed by Celartem, Inc.) if needed.

The thus-obtained digital synchronization data may be stored in, for example, a magnetic tape, a magnetic disk, CD-ROM, MO, DVD-R.

Measurement of the diameters of eyeground artery and vein on the basis of the thus-obtained digital synchronization data are performed by extracting a stationary image; i.e., digital data per frame, from the synchronization data. Specifically, a stationary eyeground image date at an arbitrary point in time “t” of an electrocardiographic signal, a fingertip sphygmographic signal, an eyepit sphygmographic signal, a temporal artery sphygmographic signal, a carotid artery sphygmographic signal, or a signal of a pulse oximeter, is extracted from motion eyeground image data, and another stationary eyeground image data at another point in time after an appropriate interval “t+Δt” is extracted from the motion eyeground image data, and the amount of change per unit time in the eyeground artery or vein diameter is calculated on the basis of these stationary eyeground image data (As described above, the time “t” is preferably determined in accordance with an electrocardiographic signal, a fingertip sphygmographic signal, an eyepit sphygmographic signal, a temporal artery sphygmographic signal, a carotid artery sphygmographic signal, or a signal of a pulse oximeter which has been synchronized with a motion eyeground image.).

Specifically, when the eyeground artery or vein diameter at the time “t” is represented by “r₁,” and the eyeground artery or vein diameter at the time “t+Δt” is represented by “r₂,” the amount of change in the eyeground artery or vein diameter per unit time “Δt”; i.e., Δr, can be calculated by use of the following formula:

Δr=|r₁−r₂|/Δt

The amount of change Δr (speed pulse wave) can be obtained in this way.

The element of the acceleration pulse wave can be calculated by further differentiation of said Δr with respect to the time.

When stationary image data are extracted from digital synchronization data of motion eyeground image data and an electrocardiographic signal, a fingertip sphygmographic signal, an eyepit sphygmographic signal, a temporal artery sphygmographic signal, a carotid artery sphygmographic signal, or a pulse oximeter signal data while the motion eyeground image and the electrocardiogram are displayed on display means (e.g., a computer display) of a computer terminal, extraction operation can be visualized. Such visualization of extraction operation is preferred. Therefore, the present software preferably includes visualization means on display means of a computer terminal.

Obviously, the present software preferably includes means for detecting difference between the eyeground artery and vein diameters in an eyeground image in synchronization with an electrocardiographic signal, a fingertip sphygmographic signal, an eyepit sphygmographic signal, a temporal artery sphygmographic signal, a carotid artery sphygmographic signal, or a pulse oximeter signal at an arbitrary point in time, more specifically, means for calculating each of the target eyeground artery and vein diameters on the basis of the eyeground images in synchronization with different electrocardiographic signals, fingertip sphygmographic signals, eyepit sphygmographic signals, temporal artery sphygmographic signals, carotid artery sphygmographic signals, or pulse oximeter signals, and calculating the change in each of the eyeground artery and vain diameters per unit time and in addition the inflection point in the change, and, on the basis of these data, detecting the waveform difference between the volume pulse waves of the eyeground artery and vein.

The present software preferably includes means for detecting the degree of senescence of the capillary by correlating the component difference between the volume pulse waves of the eyeground artery and vein with the peripheral vessel resistance. It is capable and preferable that the present software includes a program for executing processing that, when the component difference of a subject are lower than that of the average value on the preliminarily set condition (e.g. age and sexuality), it is considered that the peripheral vessel resistance is also lower, namely that the stenosis of the capillary is not severer than average, or the extensibility of the capillary is maintained, and, on the contrary, when the component difference of an subject are higher than that of the average value on the preliminarily set condition (e.g. age and sexuality), it is considered that the peripheral vessel resistance is also higher, namely that the stenosis of the capillary is severer than average, or the extensibility of the capillary is not maintained.

The present software can be written by developing a desired algorithm with use of a typical computer programming language.

Examples of the computer programming language which may be employed include low-level languages such as a machine language and an assembly language; high-level languages such as Fortran, ALGOL, COBOL, C, BASIC, PL/I, Pascal, LISP, Prolog, APL, Ada, Smalltalk, C++, and Java (registered trademark); fourth generation languages; and end user languages. If desired, special purpose languages may be employed.

The present invention also provides a computer program comprising an algorithm for executing the present software. The present invention also provides an electronic medium containing the present software, which is executed by means of the computer program.

No particular limitations are imposed on the electronic medium in which the present software can be stored. Examples of the electronic medium which may be employed include a magnetic tape, a magnetic disk, CD-ROM, MO, and DVD-R.

In the extremely preferred mode of the present detection system, a blood pressure detecting means for detecting a blood pressure as a parameter is provided together with the means for detecting the waveform difference between the volume pulse waves of the eyeground artery and vein. The intensity of a pulse wave tends to depend on the height of blood pressure. Namely, as for the same person, the higher the blood pressure is, the greater the amplitude of the volume pulse wave tends to be, and the lower the blood pressure is, the narrower the amplitude tends to be. Therefore, when the present detection system is applied to a subject, it is extremely important to detect the blood pressure of the subject over time during detecting the waveform difference in volume pulse wave (preferably, the component difference in acceleration pulse wave), in order to ensure the accuracy of the results of the present detection system. In addition, the degree of extensibility (flexibility) of a peripheral vessel can bet detected by correlating the waveform differences between the volume pulse waves of the eyeground artery and vein at different blood pressures of a subject. Comparing the case that the extensibility of a peripheral vessel is excellent with the case that the extensibility is inferior, in the case that the extensibility is excellent, the waveform differences between the volume pulse waves at a prescribed different blood pressures is smaller due to absorption by the excellently extensive peripheral vessel, and, on the contrary, in the case that the extensibility is poor, the waveform differences at a prescribed different blood pressures is greater because stress by waveform differences between the volume pulse waves is not sufficiently absorbed in the peripheral vessel. In this way, by grasping the waveform differences between the volume pulse waves at different blood pressures, the extensibility of the peripheral vessel can be detected and, on the basis of the extensibility of the peripheral vessel, the degree of senescence of the whole cardiovascular system of the body can be grasped.

As a blood pressure detecting means, a blood-pressure meter may be employed. As a blood-pressure meter, mercury, aneroid, and electronic meter may be employed. Anyway, it is preferable that a blood-pressure meter can measure the blood pressure not intermittently but continuously (measure the waveform continuously).

### Effects of the Invention

As described above, according to the present detection system, by using the waveform difference between the volume pulse waves of the eyeground artery and vein (preferably, the component difference between the acceleration pulse waves based on the volume pulse waves) as a parameter, the degree of senescence of the capillary of the body, and furthermore the degree of the whole cardiovascular system of the body, can be detected, for example, as a rate of variation with time or a vascular age.

### BRIEF DESCRIPTION OF DRAWINGS

FIG. 1 is a block diagram showing the configuration of an embodiment of the present detection system.

FIG. 2A shows the first part of an embodiment of a flowchart on the basis of the algorithm of the present software.

FIG. 2B shows the second part of an embodiment of a flowchart on the basis of the algorithm of the present software.

FIG. 2C shows the third part of an embodiment of flowchart on the basis of the algorithm of the present software.

### BEST MODE FOR CARRYING OUT THE INVENTION

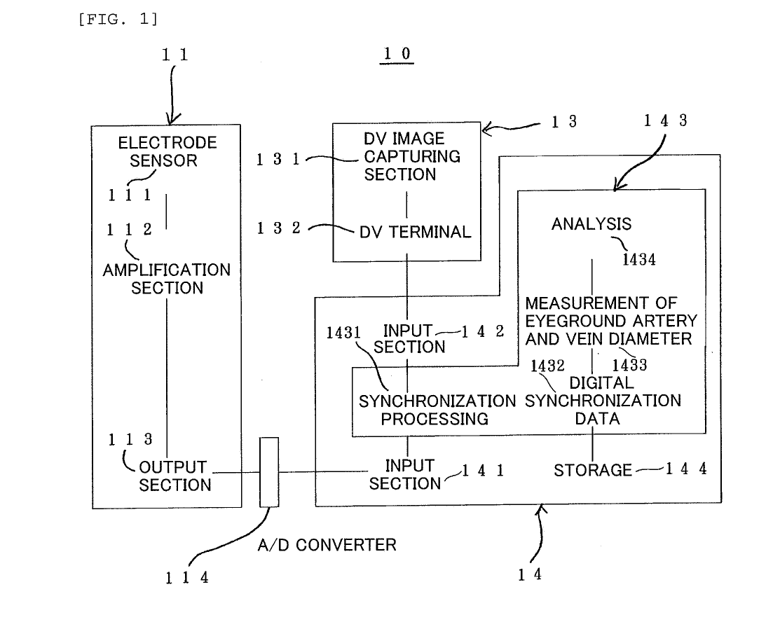

FIG. 1 is a block diagram showing the configuration of an embodiment of the present detection system.

FIG. 1 shows the detection system 10 which is one of the best embodiments of the present detection system. In the detection system 10, synchronization of an eyeground image with “an electrocardiographic signal, a fingertip sphygmographic signal, an eyepit sphygmographic signal, a temporal artery sphygmographic signal, a carotid artery sphygmographic signal, or a pulse oximeter signal (hereinafter these signals may be referred to as a “biosignal”)” and so on are carried out on the computer 14.

In the detection system 10, in order to execute the synchronizing process on the computer 14, the biosignal is input directly from the output section 113 of the biosignal detection unit 11 toward the input section 141 of the computer 14. It is preferable that the biosignal is digitized by the A/D conversion mechanism 114 and so on.

In the eyeground image detecting unit 13, the eyeground image of a subject is captured through the DV image capturing section 131, which corresponds to the imaging section of a digital video camera, the motion eyeground image signal is extracted from the above captured image, and this motion signal is input through the DV terminal 132 into the input section 142 of the computer 14 through the DV capture cards or the like. It is suitable that the digital video camera of the DV image capturing section 131 has as high resolution as possible, in order to measure a delicate difference between the eyeground artery diameter and the eyeground vein diameter. Specifically, it is preferred that the digital video camera has a resolution of more than 8,000,000 pixels. It is obvious that the DV image capturing section 131 is provided with mechanisms for capturing the eyeground of a subject, with which conventional eyeground cameras are provided, such as, for example, eye lens, a source of light, mechanism to align, field angle adjustment mechanisms, if necessary.

The motion eyeground image signal input into the computer 14 and the biosignal input from the input section 141 are combined in parallel so as to synchronize the motion eyeground image data with the biosignal in every frame (synchronization processing 1431), thereby obtain a digital synchronization data 1432 of the motion eyeground image data and the biosignal. The digital synchronization data 1432 may be subjected to compression or the like as needed.

As described above, the synchronization data 1432 may be directly employed in a subsequent step, for example, a step for measuring diameters of eyeground artery and vein. Alternatively, the synchronization data 1432 may be temporarily stored in an electronic medium (144).

In an eyeground artery and vein diameter measurement step 1433, one or more target sites (the paired eyeground artery and vein traveling in the same direction) is selected on the basis of the synchronization data 1432 (preferably, the vicinity of the optic papilla of the eyeground vain is selected as one of the target sites), and the diameters of the eyeground artery and vein at the target site(s) are measured. The eyeground artery diameters and the eyeground vein diameters are measured at the target sites and at different timings. The timings may be arbitrarily determined, so long as difference between the thus-obtained eyeground vein images can be detected (preferably, the timings are determined so as to depend on a biosignal).

In an analysis step 1434, the difference between the eyeground artery diameters and the difference between the eyeground vein diameters at every target site and at different timings are measured on the basis of the eyeground artery and vein diameters measured in the eyeground artery measurement step 1433, to calculate the difference between the eyeground artery diameters per unit time and the difference between the eyeground vein diameters per unit time depending on a biosignal (“the speed pulse wave”) or calculate the acceleration pulse wave on the basis of the speed pulse wave. When an interval “ΔT” capable of obtaining different image frames in which difference between thy eyeground artery images and difference between the eyeground vein images can be clearly detected, is determined as an interval between the above different timings, the differences of the eyeground artery and vein diameters per unit time can be calculated. When a biosignal at every timing is identified, the differences of the eyeground artery and vein diameters can be connected to the biosignal.

The waveform difference between the volume pulse waves of paired eyeground artery and vein traveling in the same direction or the component difference in acceleration pulse waves are detected on the basis of the difference between the eyeground artery and vein diameters per unit time depending on a biosignal (the speed pulse wave) or the acceleration pulse waves which have been calculated in the analysis step 1434, and, the peripheral vessel resistance of a subject can be detected by using the waveform difference or the component difference as an index. The fact that the peripheral vessel resistance of the eyeground is high means that the peripheral vessel resistance of whole body is high, namely that the finer vessels in internal organs and tissues (capillary in internal organs) is stenosed severely and less extensible (less flexible), which leads to the senescence of the whole cardiovascular system. Besides, more reliable and comprehensive degree of senescence can be provided when considering other existing indexes capable of representing degree of senescence, for example, the change in an MRI image signal on head or histopathological evaluation, in conjunction with the data provided by the present detection system.

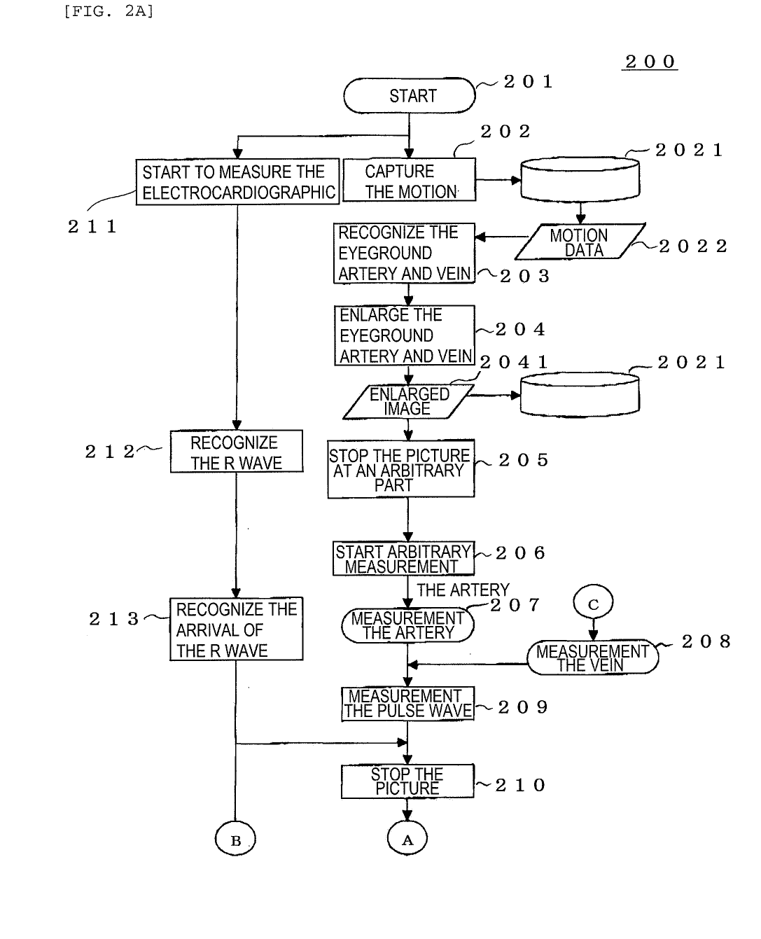

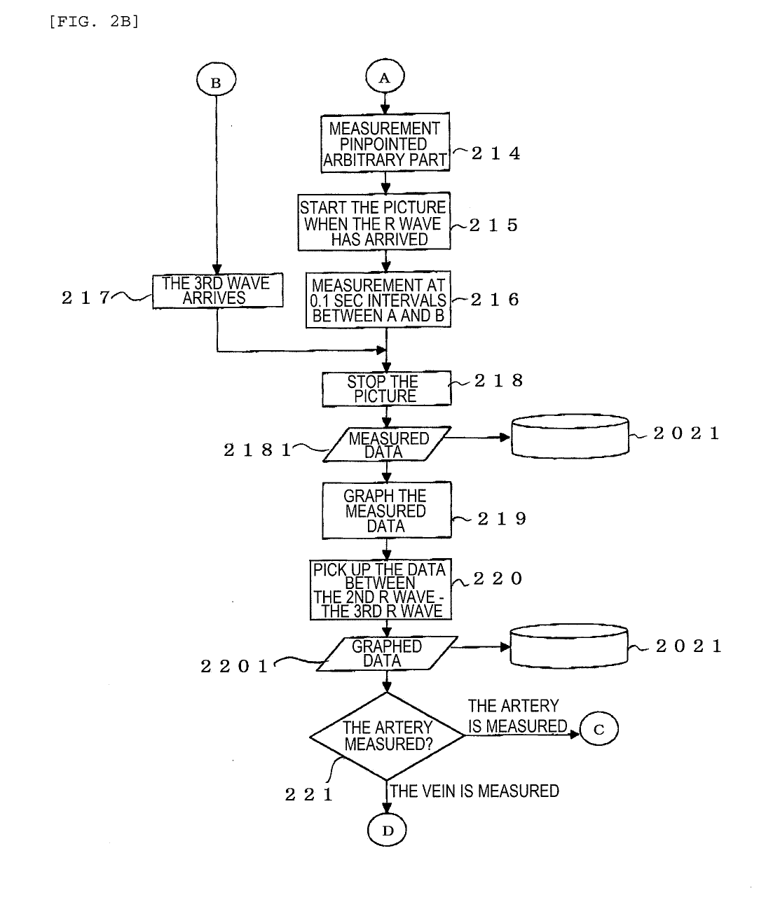

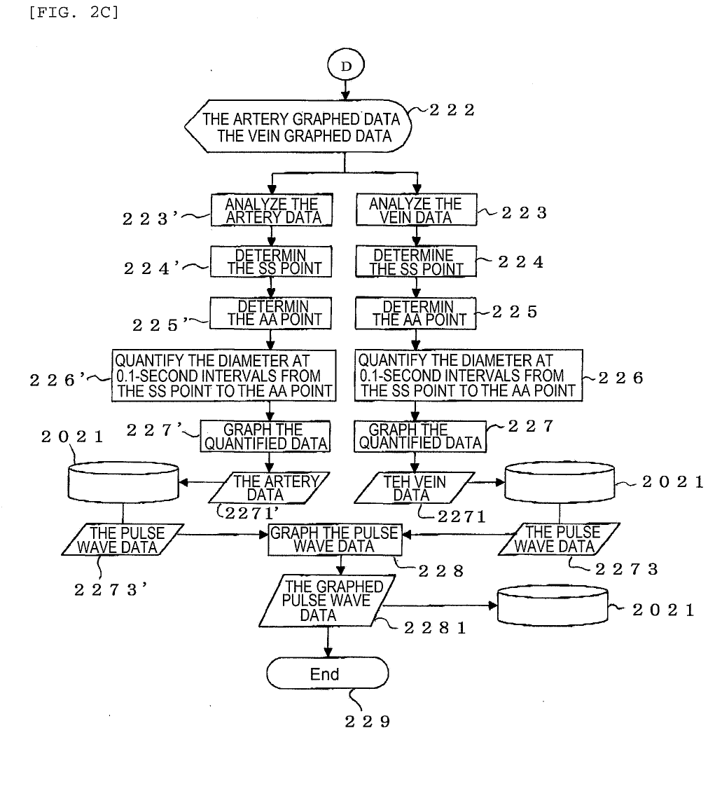

FIGS. 2A through 2C show an example of a flowchart (200) on the basis of the algorithm of the present software employed in a processing unit of the computer 14 of the present detection system 10. In this example, motion pictures are used, but continuous still images, for example, can be also used alternatively. The data of one still image in the present software corresponds to the data of one frame of the motion picture in this embodiment.

As shown in FIG. 2A, in the step 201 “start,” the computer 14 is set up such that the present software can execute the process shown in the flowchart 200. In a step 202 “MOTION CAPTURING”, the eyeground motion picture is captured through the DV image capturing section 131 (corresponding to image capturing section in a digital video camera). The motion eyeground images captured by the DV image capturing section 131 are stored in the hard disk 2021 of the computer 14. And then, the intended eyeground motion picture data 2022 are re-called up from the hard disk 2021 and checked on the display of the computer 14, the paired eyeground artery and vein traveling in the same direction are recognized and the part is pointed out (step 203), and the pointed part in the eyeground motion picture are enlarged (step 204). The enlarged image data 2041 are stored in the hard disk 2021. And then, the eyeground motion picture is stopped at an arbitrary part of the enlarged image (a part of the pointed part, in which a vessel diameter can be adequately confirmed) (step 205), and then the measurement at this arbitrary part is performed (step 206).

Next, measurement of an eyeground artery (or an eyeground vein: step 208) at the above-described arbitrary part is carried out (step 207). Firstly, a pulse wave is measured (step 209). In this step, on the basis of the change of the eyeground artery (or eyeground vein) diameter in the motion picture, the pulse wave of the eyeground artery (or eyeground vein) is grasped. It is preferable that the pulse wave grasped in this step is synchronized with a biosignal or blood pressure variations (not figured). The biosignal is measured (step 211) in parallel with the motion capturing step 202. For example, a certain peak (e.g., an R wave) is recognized in an electrocardiographic signal, which is one of biosignals (step 212), the arrival of this peak (e.g., an R wave) at the computer 14 is confirmed on the display, for example (step 213), the pulse wave measured at the step 209 is synchronized with the certain peak in the electrocardiographic signal, and then the eyeground motion picture is stopped (step 210) (flowchart connector A).

The flowchart connector A in FIG. 2B is corresponding to that shown in FIG. 2A. Pinpoint measurement of the pointed part of the eyeground artery or vein traveling in the same direction shown on the display is carried out on the condition that the pulse wave is synchronized with the biosignal by the above stopping step 210 (step 214). In the measurement step, the eyeground motion picture is re-started when the R wave has arrived (step 215). Next the volume pulse waves are measured at e.g. 0.1 sec intervals between two certain point (between point A and point B) in one of the paired eyeground artery and vein traveling in the same direction (step 216). Next, after the flowchart connector B, the 3rd wave in the electrocardiographic signal is recognized (step 217), and then the eyeground motion picture is stopped by the 3rd wave signal, i.e., when the pulse wave is synchronized with the 3rd wave (step 218). The measured data 2181 at this timing are stored in the hard disk 2021. Next the measured data 2181 are graphed (step 219). Certain data are picked up from this graphed data (step 220) (the graphed data between the 2nd R wave and the 3rd R wave are employed in this embodiment), and the picked-up data 2201 are stored in the hard disk 2021.

Next it is judged whether the measured vessel is an artery or a vein (step 221). When the artery has been measured, the process returns through the flowchart connector C to the preliminary step toward step 209, and the steps subsequent to the pulse wave measuring step 209 as described above are carried out on the eyeground vein (the return process about an eyeground vein is in the aforementioned parenthetic reference). When the vein has been measured (in this embodiment, the data of the eyeground artery have been obtained in advance of process of obtaining the data of the eyeground vein), the process goes through the flowchart connector D to the next FIG. 2C. At this point, the graphed data of the eyeground artery and vein need to be both obtained (step 222). Next these graphed data of the eyeground artery and vein are analyzed (step 223) (step 223′), the timings, when the diameters of the eyeground artery and vein are minimum, and the minimum diameters (SS point) are determined (step 224) (step 224′). The timings, when the diameters of the eyeground artery and vein are maximum and are forward and nearest from the SS point, and the maximum diameters (AA point) are determined (step 225) (step 225′). Next the measured values of the diameter of the blood vessel are quantified at 0.1-second intervals from the SS point to the AA point (step 226) (step 226′). Next this quantified data are graphed (step 227) (step 227′). The graphed quantified data 2271 (2271′) of the eyeground artery and vein are stored in the hard disk 2021. The stored data 2273 (2273′) of the eyeground artery and vein are graphed as pulse wave data (step 228). This graphed pulse wave data 2281, including electric data not only of the volume pulse wave figures but also of the speed pulse wave figures and the acceleration pulse wave figures, are stored in the hard disk 2021 and the series of process end at the end terminal 229. As for the volume pulse wave figures and the speed pulse wave figures of the stored pulse wave data, the degree of senescence of the capillary system of the eyeground or the whole body can be detected by comparing the waveforms of the eyeground artery and vein traveling in the same direction, in view of the fact that the more different the waveforms are, the higher the peripheral vascular resistance is at the eyeground. As for the acceleration pulse wave figures, the degree of senescence can be detected by calculating b wave /a wave or d wave /a wave and calculating the component difference of the volume pulse waves between the eyeground artery and vein about these waves.

The aforementioned software may include, for example, an additional function for displaying data on the above-measured component difference of the acceleration pulse wave between the eyeground artery and vein in parallel with normalized data (e.g., normalized data obtained by normalizing, with respect to age or sex, the component difference of the acceleration pulse wave between the eyeground artery and vein), and for comparing the eyeground artery and vein diameter data with the normalized data. Specifically, the software may include an additional function for displaying data on the above-measured eyeground artery and vein diameter of a subject on the display of the computer 14, and for comparing the eyeground artery and vein diameter data with the normalized data. Specifically, the software may include an additional function for displaying the above-calculated values in parallel with the normalized data, and for calculating the deviation of the calculated values on the basis of the normalized data. The results of the above comparison can be stored as data on the subject.

The aforementioned software may further include, as above mentioned, an additional function for calculating the peripheral vascular resistance at the certain blood pressure, correlating the calculated values with the blood pressure data of a subject. Furthermore the software may include an additional function for detecting the extensibility of the peripheral vascular of the subject, by calculating the peripheral vascular resistance at different blood pressures by increasing or decreasing the blood pressure of the subject with a hypotensor, a vasopressor, a shakedown or a hypnogenesis.

As described above, the algorithm can be programmed by use of a typical computer programming language.

1. A blood vessel senescence detection system comprising:
a volume pulse waves detecting means for detecting volume pulse waves of paired eyeground artery and vein traveling in a same direction; and
a peripheral vascular resistance detecting means for detecting peripheral vascular resistance by using waveform difference between the volume pulse wave of the eyeground artery and the volume pulse wave of the eyeground vein as a parameter.

2. The blood vessel senescence detection system according to claim 1, wherein the waveform difference between the volume pulse wave of the eyeground artery and the volume pulse wave of vein is component difference between an acceleration pulse wave of the eyeground artery and an acceleration pulse wave of the eyeground vein, the acceleration pulse waves being caused by propagation of the volume pulse waves.

3. The blood vessel senescence detection system according to claim 1, wherein the waveform difference between the volume pulse wave of the eyeground artery and the volume pulse wave of the eyeground vein is detected by using images of the eyeground artery and vein taken by an eyeground camera, an ultrasound Doppler method, or a laser Doppler method.

4. The blood vessel senescence detection system according to claim 1, wherein the volume pulse waves of the eyeground artery and vein are detected in synchronization with a biosignal selected from the group consisting of an electrocardiographic signal, a fingertip sphygmographic signal, an eyepit sphygmographic signal, a temporal artery sphygmographic signal, a carotid artery sphygmographic signal, and a pulse oximeter signal.

5. The blood vessel senescence detection system according to claim 1, further comprising:
a component difference detecting means for detecting component difference between the volume pulse wave of the eyeground artery and the volume pulse wave of the eyeground vein; and
a blood pressure detecting means for detecting a blood pressure.

6. The blood vessel senescence detection system according to claim 5, further comprising:
a means for detecting extensibility of a peripheral vessel by correlating the component differences between the volume pulse wave of the eyeground artery and the volume pulse wave of the eyeground vein at different blood pressures.

7. The blood vessel senescence detection system according to claim 1, wherein progression of blood vessel senescence is expressed as increase of a rate of change of arteriosclerosis with time.
